(19) United States
(12) Patent Application Publication
(10) Pub. No.: US 2024/0328921 A1
(43) Pub. Date: Oct. 3, 2024

(54) METHOD FOR DETERMINING EFFECTIVE STORAGE CAPACITY OF GAS STORAGE RECONSTRUCTED FROM WATER-FLOODED VOLATILE OIL RESERVOIR

(71) Applicant: Northeast Petroleum University, Daqing (CN)

(72) Inventors: Junchang SUN, Daqing (CN); Jieming WANG, Daqing (CN); Huayin ZHU, Daqing (CN); Xiaofei FU, Daqing (CN); Wei LIU, Daqing (CN); Chun LI, Daqing (CN); Lingdong MENG, Daqing (CN); Guangliang GAO, Daqing (CN); Yanchun SUN, Daqing (CN); Kun TU, Daqing (CN); Lin SHANG, Daqing (CN); Shanpo JIA, Daqing (CN); Shaojing ZHENG, Daqing (CN); Haiyan HE, Daqing (CN); Runya SHEN, Daqing (CN); Xiaohu GUO, Daqing (CN); Ruotong CHEN, Daqing (CN)

(73) Assignee: Northeast Petroleum University, Daqing (CN)

(21) Appl. No.: 18/618,506

(22) Filed: Mar. 27, 2024

(30) Foreign Application Priority Data
Mar. 30, 2023 (CN) 202310325083.0

(51) Int. Cl. G01N 15/08; E21B 43/16; E21B 43/20; E21B 49/08; G01N 33/24

(52) U.S. Cl. CPC G01N 15/088; E21B 43/166; E21B 49/087; G01N 33/24; E21B 43/20

(57) ABSTRACT

The present disclosure provides a method for determining effective storage capacity of a gas storage reconstructed from a water-flooded volatile oil reservoir. The method includes: determining an oil reservoir water flooding recovery ratio; determining crude oil production extracted only due to a water flooding effect in an oil reservoir development process; determining an oil-bearing pore volume of a flooded zone of a reservoir influenced due to the water flooding effect and an oil-bearing pore volume of a pure oil zone of a reservoir not influenced due to the water flooding effect corresponding to its original state before an oil reservoir is put into development when the gas storage is reconstructed from the oil reservoir; determining an effective pore volume for gas storage; and computing the effective storage capacity of the gas storage reconstructed from the water-flooded volatile oil reservoir.

### CROSS REFERENCE TO RELATED APPLICATION

This patent application claims the benefit and priority of Chinese Patent Application No. 202310325083.0, filed with the China National Intellectual Property Administration on Mar. 30, 2023, the disclosure of which is incorporated by reference herein in its entirety as part of the present application.

### TECHNICAL FIELD

The present disclosure relates to the technical field of underground storage of natural gas, and particularly relates to a method for determining effective storage capacity of a gas storage reconstructed from a water-flooded volatile oil reservoir.

### BACKGROUND

As the most effective facility for large-scale safe storage of natural gas in the world at present, an underground gas storage (hereinafter referred to as a gas storage) generally stores gas and regulates a peak through “injection in summer and extraction in winter”. That is, balance of supply and demand is adjusted by injecting and storing natural gas in a low period of gas consumption in the summer market and extracting natural gas in a peak period of gas consumption in the winter market. It has functions of seasonal peak regulation, accident emergency and strategic reserve. There are many different types of the gas storage, including a gas reservoir, an oil reservoir, a salt cavern, an aquifer, and a pit.

Effective storage capacity is one of the core parameters for evaluation of peak regulation and gas storage abilities of the gas storage. A gas storage reconstructed from a gas reservoir has its inherent gas storage space that objectively exists, so it can be evaluated more accurately through geological research and dynamic analysis of gas reservoir development. At present, a method for predicting effective storage capacity of a gas storage reconstructed from a gas reservoir by considering water invasion, stress sensitivity and other factors has been provided. Reconstruction of the gas storage from the oil reservoir is completely different from reconstruction of the gas storage from the gas reservoir. Because pore of reservoir rocks are almost filled with liquid such as residual oil, natural invasion formation water and/or artificially injected water before gas injection, reconstruction of the gas storage from the oil reservoir needs to achieve gas-liquid “space replacement” through long-term circulating gas driven oil recovery and liquid discharge. In this way, a new gas storage space is created from scratch. The form scale of storage capacity is restricted by utilization efficiency of gas injection driven oil displacement and liquid discharge. Meanwhile, the oil reservoir is generally subjected to natural water driving and/or artificial water injection before reconstructed into the gas storage. These operations often lead to a high degree of water flooding in middle and lower parts of a reservoir and less influence or no water invasion on a top of the reservoir. Especially, oil reservoirs greater in reservoir thickness and/or larger in structural dip angle have remarkable fluid zoning features of “top oil and bottom liquid (oil+water)” in their development process. Therefore, when a gas storage is reconstructed from a flooded oil reservoir through gas injection, gas may displace oil on the top of the reservoir and gas may displace liquid (oil+water) on a bottom of the reservoir. Because of different fluids in different zones, a sweep degree and displacement efficiency of injected gas will obviously vary.

In addition, gas injected into the gas storage is generally purified natural gas transported via long-distance pipes. Its methane content is high (generally greater than 90%). This gas is referred to as dry gas. During gas driven oil recovery and liquid discharge through circulating injection-production when the gas storage is reconstructed from the oil reservoir, complex phase behaviors such as component exchange and interphase mass transfer occur after injected gas makes contact with crude oil. These behaviors continuously change crude oil properties, and especially for a volatile reservoir greater in intermediate hydrocarbon content, smaller in viscosity and better in oil properties. This kind of volatile reservoirs is the optimal oil reservoir type for reconstruction of the gas storage. An interphase mass transfer effect between injected gas and crude oil is more obvious while the gas storage is reconstructed from the water-flooded volatile oil reservoir through gas injection. The injected gas constantly extracts intermediate hydrocarbon greater in content in crude oil. In this way, contents of heavy components such as C7+in remaining crude oil of the oil reservoir increase constantly, leading to increase in crude oil density and volume shrinkage. Further, fluid distribution features and occupied space sizes of pore in the reservoir of the oil reservoir are changed.

Therefore, when the effective storage capacity of the gas storage reconstructed from the water-flooded volatile oil reservoir is predicted, not only the fluid zoning features of the pore in the reservoir but also influence of the complex phase behaviors between the injected gas and the crude oil on the fluid distribution features and occupied space sizes of pore in the reservoir needs to be considered. However, no method is currently provided for predicting effective storage capacity by considering fluid distribution differences of the reservoir of the water-flooded volatile oil reservoir and the complex phase behaviors between the injected gas and the crude oil. (For example, the article “New method for predicting a maximum working gas volume of an underground gas storage reconstructed from a sandstone reservoir” in “Natural Gas Geoscience”, Volume 16, No.5, October 2005, and the article “Method for computing storage capacity of a gas storage reconstructed from a sandstone gas-cap reservoir” in “Natural Gas Industry”, Volume 27, No.11, November 2007). Both of these methods only consider macroscopic gas-oil interface migration or generally consider a reservoir fluid distribution as a flooded zone. This kind of method is only suitable for conventional black oil reservoirs whose reservoir strata are completely flooded. If they are applied to volatile oil reservoirs and partially flooded oil reservoirs (including conventional black oil and volatile oil reservoirs), a larger error in computed effective storage capacity will be caused. The more obvious the fluid zoning features of the reservoir of the oil reservoir, the stronger volatility of the crude oil, and the larger errors in predicting the effective storage capacity of the gas storage reconstructed from the oil reservoir through the above methods.

### SUMMARY

In view of the above problems, an objective of the present disclosure is to provide a method for determining effective storage capacity of a gas storage reconstructed from a water-flooded volatile oil reservoir, so as to obtain more accurate effective storage capacity of the gas storage reconstructed from the water-flooded volatile oil reservoir. In this way, problems that an existing method in the background art does not distinguish between a flooded zone and a single oil zone of a reservoir when predicting effective storage capacity of a gas storage, and does not consider influence of complex phase behaviors between injected gas and crude oil on fluid distribution of the reservoir so as to cause large errors in effective storage capacity are solved. The method is different from a conventional method in that, according to fluid occurrence characteristics of the reservoir of the water-flooded volatile oil reservoir, a fluid distribution zone of the reservoir is divided into a flooded zone and a single oil zone of the reservoir, and then oil-bearing pore volumes of the flooded zone and the single oil zone of the reservoir in an original state before the oil reservoir is put into development are accurately obtained through inverse computation according to dynamic data such as crude oil production extracted only due to a water flooding effect in an oil reservoir development process and a water drive recovery ratio. In this way, a difference in spatial efficiency of gas storage pore formed by gas injection displacement between different fluid zones of the reservoir of the gas storage reconstructed from the flooded oil reservoir can be accurately reflected, and errors in predicting the effective storage capacity through the conventional method are greatly reduced. Meanwhile, in view of a strong interphase mass transfer effect between volatile oil and injected gas, changes of properties of remaining oil of the oil reservoir caused by the complex phase behaviors between the injected gas and the crude oil in a process of reconstructing the gas storage from the flooded oil reservoir are further considered, such that an effective gas storage pore space can be generated due to a shrinkage effect of the crude oil. The method overcomes defects of the conventional method that the flooded zone and the single oil zone of the reservoir are not distinguished and oil-bearing pore volumes in different zones cannot be accurately computed when the effective storage capacity of the flooded oil reservoir is designed and influence of complex phase behaviors between the injected gas and the crude oil on an effective gas storage space of the gas storage is not considered, and greatly enhances scientificity and accuracy of a design of the effective storage capacity of the gas storage reconstructed from the water-flooded volatile oil reservoir.

The present disclosure provides the technical solution as follows: a method for determining effective storage capacity of a gas storage reconstructed from a water-flooded volatile oil reservoir, including the following steps:
step 1: determining an oil reservoir water drive recovery ratio according to dynamic oil reservoir development data;
step 2: determining crude oil production extracted only due to a water flooding effect in an oil reservoir development process according to the dynamic oil reservoir development data;
step 3: determining an oil-bearing pore volume of a flooded zone of a reservoir influenced due to the water flooding effect in an original state before an oil reservoir is put into development when the gas storage is reconstructed from the oil reservoir according to the oil reservoir water drive recovery ratio and crude oil production extracted only due to the water flooding effect;
step 4: determining an oil-bearing pore volume of a single oil zone of a reservoir not influenced due to the water flooding effect in an original state before the oil reservoir is put into development when the gas storage is reconstructed from the oil reservoir according to a total original oil-bearing pore volume before the oil reservoir is put into development and the oil-bearing pore volume of the flooded zone of the reservoir in the original state before the oil reservoir is put into development;
step 5: determining an effective pore volume for gas storage produced in the flooded zone and the single oil zone of the reservoir due to a gas injection driven oil recovery and liquid discharge displacement effect according to macro sweep coefficients and micro displacement efficiency of injected gas in the flooded zone and the single oil zone of the reservoir separately when the gas storage is reconstructed from the oil reservoir;
step 6: determining an effective pore volume for gas storage produced in the single oil zone of the reservoir of the gas storage reconstructed from the oil reservoir due to a volume shrinkage effect of final remaining oil according to crude oil property change features caused by multi-cycle injection-production interphase mass transfer of the gas storage reconstructed from the oil reservoir; and
step 7: computing the effective storage capacity of the gas storage reconstructed from the water-flooded volatile oil reservoir according to a total effective pore volume for gas storage produced due to the gas injection driven oil recovery and liquid discharge displacement effect and the volume shrinkage effect of the final remaining oil in the single oil zone of the reservoir.

The determining an oil reservoir water drive recovery ratio includes: conducting computation on the basis of data of oil production and water production in the oil reservoir development process according to a formula E_{w}=(1/B₁)×[1g(21.28/B₁−A₁)]/N.

E_{w} denotes the oil reservoir water drive recovery ratio. A₁ and B₁ denote an intercept and a slope obtained through mathematical fitting of a linear functional relation between cumulative oil production and cumulative water production in an oil reservoir water drive development process in a semi-logarithmic coordinate system, respectively. N denotes a dynamic reserve of the oil reservoir.

The determining crude oil production extracted only due to a water flooding effect in an oil reservoir development process includes: conducting computation on the basis of parameter data of oil production, water production, water influx, a water injection amount and a crude oil property in the oil reservoir development process according to a formula

$${N}_{pw} = {\sum }_{j = 1}^{n} {N}_{pj} \times [ ( {W}_{ej} + {W}_{ij} {B}_{wj} ) / ( {N}_{pj} {B}_{oj} + {W}_{pj} {B}_{wj} ) ] .$$

N_{pw} denotes crude oil production extracted only due to the water flooding effect in the oil reservoir development process. Further, j denotes different time points in monthly units in the oil reservoir development process. Further, n denotes a total development and extraction month number before the gas storage is reconstructed from the oil reservoir. Nₚⱼ, Wₑⱼ, Wᵢⱼ and Wₚⱼ denote oil production, natural water influx, an artificial water injection amount and water production in different months, respectively. B_{wj} and Bₒⱼ denote a formation water volume coefficient and a crude oil formation volume factor under average formation pressure in different months, respectively.

The determining an oil-bearing pore volume of a flooded zone of a reservoir influenced due to the water flooding effect in an original state before an oil reservoir is put into development when the gas storage is reconstructed from the oil reservoir includes: conducting computation according to a formula V_{wz}=(N_{pw}×Bₒᵢ)/E_{w}.

V_{wz} denotes the oil-bearing pore volume of the flooded zone of the reservoir influenced due to the water flooding effect in the original state before the oil reservoir is put into development when the gas storage is reconstructed from the oil reservoir. N_{pw} denotes crude oil production extracted only due to the water flooding effect in an oil reservoir water drive development process. Bₒᵢ denotes a crude oil formation volume factor under original formation pressure of the oil reservoir.

The determining an oil-bearing pore volume of a single oil zone of a reservoir not influenced due to the water flooding effect in an original state before the oil reservoir is put into development when the gas storage is reconstructed includes: conducting computation according to a formula V_{oz}=N×Bₒᵢ−V_{wz}.

V_{oz} denotes the oil-bearing pore volume of the single oil zone of the reservoir not influenced due to the water flooding effect in the original state before the oil reservoir is put into development when the gas storage is reconstructed from the oil reservoir. N denotes a dynamic reserve of the oil reservoir. Bₒᵢ denotes a crude oil formation volume factor under original formation pressure of the oil reservoir. V_{wz} denotes the oil-bearing pore volume of the flooded zone of the reservoir influenced due to the water flooding effect in the original state before the oil reservoir is put into development when the gas storage is reconstructed from the oil reservoir.

The determining an effective pore volume for gas storage produced in the flooded zone and the single oil zone of the reservoir due to a gas injection driven oil recovery and liquid discharge displacement effect includes: conducting computation according to a formula V_{ged}=V_{wz}×η_{wz}×E_{wz}+V_{oz}×E_{oz}.

V_{ged} denotes the effective pore volume for gas storage produced in the flooded zone and the single oil zone of the reservoir due to the gas injection driven oil recovery and liquid discharge displacement effect. η_{wz} and η_{oz} denote the macro sweep coefficients of the injected gas in the flooded zone and the single oil zone of the reservoir when the gas storage is reconstructed from the oil reservoir, respectively. E_{wz} and E_{oz} denote the micro displacement efficiency of the injected gas in the flooded zone and the single oil zone of the reservoir when the gas storage is reconstructed from the oil reservoir, respectively.

The macro sweep coefficient η_{wz} of the flooded zone of the reservoir and the macro sweep coefficient η_{oz} of the single oil zone of the reservoir are computed by establishing a three-dimensional numerical simulation model of a target oil reservoir through software of Petrel RE or Eclipse and simulating a natural gas injection process of the gas storage reconstructed from the oil reservoir, or may be obtained according to an on-site pilot gas injection test of the gas storage.

The micro displacement efficiency of the flooded zone of the reservoir and the micro displacement efficiency of the single oil zone of the reservoir are obtained through the following steps: designing and developing two displacement simulation experiments of gas injection driven oil displacement and liquid discharge and gas injection driven oil displacement of a core according to fluid distribution features of the flooded zone and the single oil zone of the reservoir when the gas storage is reconstructed from the oil reservoir, respectively, and separately obtaining the micro displacement efficiency E_{wz} of the flooded zone of the reservoir and the micro displacement efficiency E_{oz} of the single oil zone of the reservoir through analysis.

The core is a regular plunger-shaped core. The core has a diameter of 2.5 cm or 3.8 cm and a corresponding length of not smaller than 5 cm or 7.2 cm, respectively.

The determining an effective pore volume for gas storage produced in the single oil zone of the reservoir of the gas storage reconstructed from the oil reservoir due to a volume shrinkage effect of final remaining oil includes: conducting computation according to a formula V_{ges}×E_{oz}×(1−η_{oz}×E_{oz})×(1−B_{os_max}/B_{oi_max}).

V_{ges} denotes the effective pore volume for gas storage produced in the single oil zone of the reservoir of the gas storage reconstructed from the oil reservoir due to the volume shrinkage effect of the final remaining oil. V_{oz} denotes the oil-bearing pore volume of the single oil zone of the reservoir not influenced due to the water flooding effect in the original state before the oil reservoir is put into development when the gas storage is reconstructed from the oil reservoir. η_{oz} denotes the macro sweep coefficient of the injected gas in the single oil zone of the reservoir when the gas storage is reconstructed from the oil reservoir. E_{oz}denotes the micro displacement efficiency of the injected gas in the single oil zone of the reservoir when the gas storage is reconstructed from the oil reservoir. B_{os_max} denotes a volume coefficient of the final remaining oil in the single oil zone of the reservoir under designed upper limit pressure after circulating injection-production of the gas storage reconstructed from the oil reservoir is stabilized. B_{oi_max} denotes a volume coefficient of original crude oil of the reservoir of the oil reservoir under the designed upper limit pressure of the gas storage.

The volume coefficient of the final remaining oil in the single oil zone of the reservoir under original formation pressure of the oil reservoir after circulating injection-production of the gas storage reconstructed from the oil reservoir is stabilized is obtained through the following steps: designing a numerical simulation or physical simulation experiment of circulating injection-production phase balance of crude oil and injected gas according to properties of the crude oil and the injected gas of the reservoir of the gas storage reconstructed from the oil reservoir and a circulating injection-production operation condition of the gas storage, and obtaining the volume coefficient B_{os_max} of the final remaining oil in the single oil zone of the reservoir under the designed upper limit pressure after circulating injection-production of the gas storage reconstructed from the oil reservoir is stabilized through simulation or testing.

The volume coefficient of the original crude oil of the reservoir of the oil reservoir under the designed upper limit pressure of the gas storage is obtained through the following step: obtaining the volume coefficient B_{oi_max} of the original crude oil of the reservoir of the oil reservoir under the designed upper limit pressure of the gas storage through a laboratory high-pressure physical property determination experiment or theoretical computation according to samples of crude oil fluid of a target oil reservoir.

The computing the effective storage capacity of the gas storage reconstructed from the water-flooded volatile oil reservoir according to a determined total effective pore volume for gas storage produced due to the gas injection driven oil recovery and liquid discharge displacement effect and the volume shrinkage effect of the final remaining oil in the single oil zone of the reservoir includes:
computing the total effective pore volume V_{ged} for gas storage produced due to the gas injection driven oil recovery and liquid discharge displacement effect and the volume shrinkage effect of the final remaining oil in the single oil zone of the reservoir according to a formula get ged ges and
computing the effective storage capacity of the gas storage reconstructed from the oil reservoir according to a formula Iₑₘₐₓ=V_{get}/B_{dg max}.

V_{ged} denotes the total effective pore volume for gas storage produced due to the gas injection driven oil recovery and liquid discharge displacement effect and the volume shrinkage effect of the final remaining oil in the single oil zone of the reservoir. V_{ged} denotes the effective pore volume for gas storage produced in the flooded zone and the single oil zone of the reservoir due to the gas injection driven oil recovery and liquid discharge displacement effect. V_{ged} denotes the effective pore volume for gas storage produced in the single oil zone of the reservoir due to the volume shrinkage effect of the final remaining oil caused by crude oil property changes. B_{dgmax} denotes a volume coefficient of the injected gas under the designed upper limit pressure of the gas storage.

The present disclosure has the following characteristics and advantages:
1. When determining the effective storage capacity of the gas storage reconstructed from the flooded oil reservoir, the existing method does not consider the flooded zone and the single oil zone formed due to the water flooding effect in the oil reservoir development process and their differences in utilization efficiency of gas injection driven displacement during storage reconstruction, and generally regards the pore in the reservoir of the gas storage reconstructed from the flooded oil reservoir as the flooded zone. Moreover, influence of complex phase behaviors such as interphase mass transfer and component exchange between injected gas and remaining oil of the reservoir of the oil reservoir on fluid distribution and occupied space sizes of the pore is not considered. Therefore, the existing method is only suitable for a design of effective storage capacity of a gas storage reconstructed from a conventional black oil reservoir having a reservoir completely flooded in a development process. When the method is applied to a design of storage capacity of a gas storage reconstructed from a conventional black oil reservoir or a volatile oil reservoir that is not completely flooded, large errors can be caused because rock pore in the flooded zone and the single oil zone of the reservoir have different gas injection utilization efficiency. According to the method, the pore of the reservoir of the flooded oil reservoir are divided into the flooded zone and the single oil zone of the reservoir, and then the oil-bearing pore volumes of the flooded zone and the single oil zone of the reservoir in the original state before the oil reservoir is put into development are accurately obtained through inverse computation according to data such as crude oil production extracted only due to the water flooding effect in the oil reservoir development process and the water drive recovery ratio. In this way, a difference in spatial efficiency of gas storage pore formed by gas injection displacement between different fluid zones of the gas storage reconstructed from the flooded oil reservoir can be accurately reflected, and errors in predicting the effective storage capacity through the conventional method are greatly reduced.
2. The present disclosure is different from the existing method in that when the effective storage capacity of the gas storage reconstructed from the water-flooded volatile oil reservoir is predicted and determined, in view of a strong interphase mass transfer effect between volatile oil and injected gas, changes of properties of remaining oil of the reservoir of the oil reservoir caused by the complex phase behaviors between the injected gas and the crude oil in a process of reconstructing the gas storage from the water-flooded volatile oil reservoir are further considered, such that an effective gas storage pore space can be generated due to a shrinkage effect of the crude oil. In this way, a defect that the existing method is only suitable for a conventional black oil reservoir without consideration of the interphase mass transfer effect between the injected gas and the crude oil is overcome, such that a formation mechanism of a gas storage space and storage capacity considered in prediction of the effective storage capacity of the gas storage reconstructed from the water-flooded volatile oil reservoir is more real and comprehensive, values of basic parameters are more accurate, and design accuracy of the effective storage capacity is higher, which provide an important scientific basis for designing of storage capacity parameters of the gas storage reconstructed from the water-flooded volatile oil reservoir and evaluation of technical and economic benefits of gas storage reconstruction.

### BRIEF DESCRIPTION OF THE DRAWINGS

The accompanying drawings described herein are provided for further understanding of the present disclosure, and constitute a part of the present disclosure, but do not constitute a limitation to the present disclosure. In the accompanying drawings:

FIG. 1 is a schematic flow diagram of a method for determining effective storage capacity of a gas storage reconstructed from a water-flooded volatile oil reservoir according to an embodiment of the present disclosure;

FIG. 2 is a schematic diagram of different fluid zones of a reservoir in a method for determining effective storage capacity of a gas storage reconstructed from a water-flooded volatile oil reservoir according to an embodiment of the present disclosure;

FIG. 3 is a schematic diagram of composition of effective gas storage pore volumes of a gas storage in a method for determining effective storage capacity of a gas storage reconstructed from a water-flooded volatile oil reservoir according to an embodiment of the present disclosure;

FIG. 4 is a mathematical fitting diagram of a linear function segment of cumulative oil production and water production in a water drive development process of a G water-flooded volatile oil reservoir according to an embodiment of the present disclosure;

FIG. 5 is a dynamic data diagram of production in a water drive development process of a G water-flooded volatile oil reservoir according to an embodiment of the present disclosure;

FIG. 6 is a diagram of a three-dimensional oil reservoir numerical simulation model established with Petrel RE software of a G water-flooded volatile oil reservoir according to an embodiment of the present disclosure;

FIG. 7 is a diagram of experimental results of pore space utilization efficiency of gas injection driven oil displacement and liquid discharge of a core in a flooded zone of a reservoir and gas injection driven oil displacement of a core in a single oil zone of a reservoir of a G water-flooded volatile oil reservoir according to an embodiment of the present disclosure;

FIG. 8 is a diagram of experimental test results of volume coefficients of remaining oil in a single oil zone of a reservoir of a gas storage reconstructed from a G water-flooded volatile oil reservoir in different injection-production cycles under designed upper limit pressure according to an embodiment of the present disclosure; and

FIG. 9 is a prediction comparison diagram of effective storage capacity of a G water-flooded volatile oil reservoir computed through different methods.

### DETAILED DESCRIPTION OF THE EMBODIMENTS

Preferred implementation modes of the present disclosure will be described in more detail below. Although the preferred implementation modes of the present disclosure are described below, it should be understood that the present disclosure may be implemented in various forms and should not be limited to the implementation modes set forth herein. On the contrary, the implementation modes are provided to make the present disclosure more thorough and complete, and to fully convey a scope of the present disclosure to those skilled in the art.

The specific implementation modes of the present disclosure will be further described in detail below with reference to the accompanying drawings.

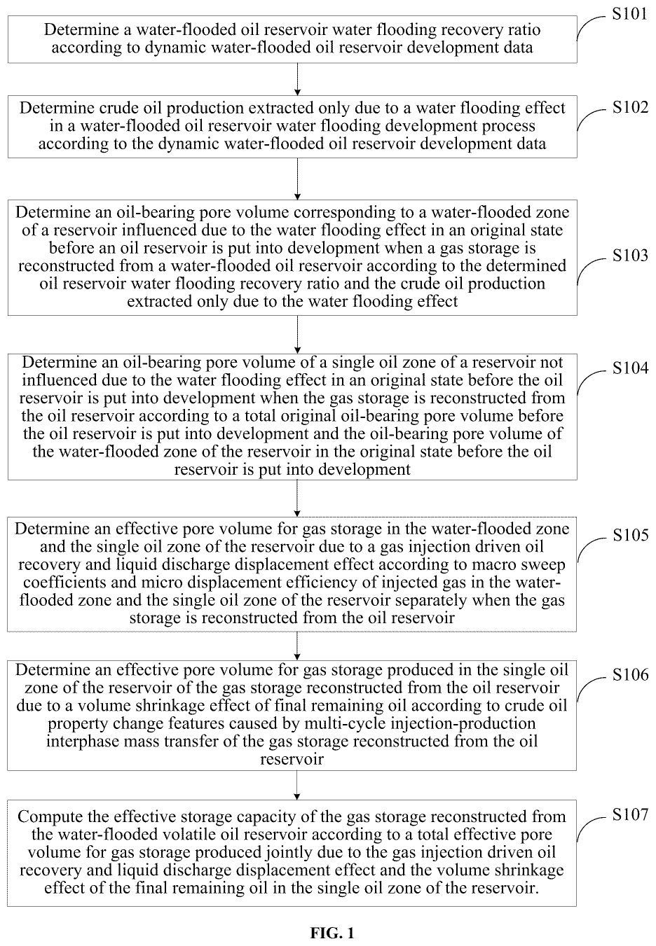

With reference to FIG. 1, a method for determining effective storage capacity of a gas storage reconstructed from a water-flooded volatile oil reservoir according to an embodiment of the present disclosure includes the following steps:

S101, an oil reservoir water drive recovery ratio is determined according to dynamic oil reservoir development data. Specifically,
computation is conducted on the basis of data such as oil production and water production in an oil reservoir development process according to a formula E_{w}=(1/B₁)×[1g(21.28/B₁−A₁)]/N.

E_{w} denotes the oil reservoir water drive recovery ratio. A₁ and B₁ denote an intercept and a slope obtained through mathematical fitting of a linear functional relation between cumulative oil production and cumulative water production in an oil reservoir water drive development process in a semi-logarithmic coordinate system, respectively. N denotes a dynamic reserve of the oil reservoir, and is analyzed and computed through a material balance method on the basis of reservoir geology and dynamic development data.

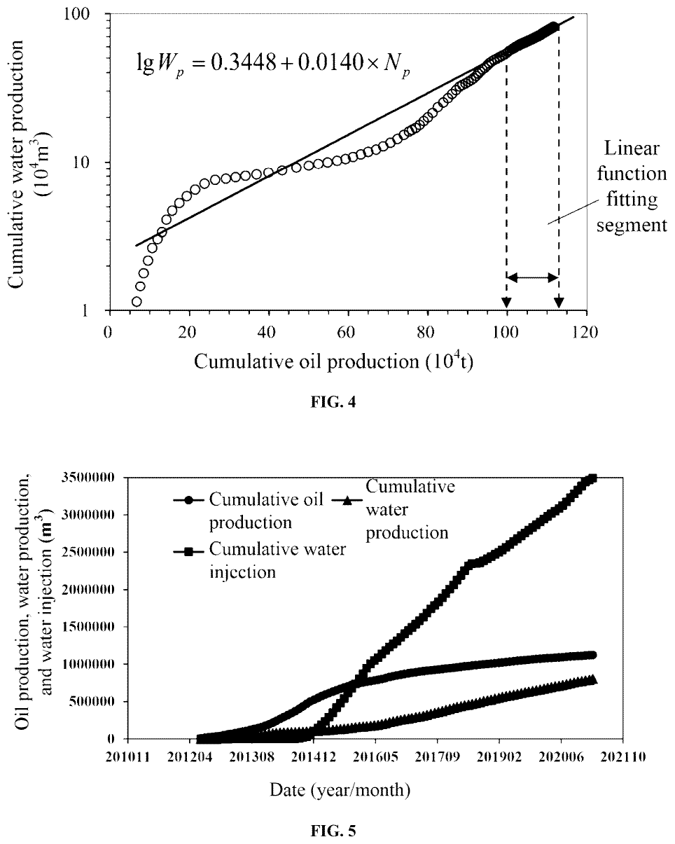

With a G water-flooded volatile oil reservoir in the Bohai Bay as an example, the oil reservoir includes five fault blocks, and it is planned to reconstruct the gas storage by preferably selecting four of the fault blocks having better geological and geographical conditions. Through material balance analysis, a total dynamic reserve of the five fault blocks of the oil reservoir is 538.0×10⁴ t. A total dynamic reserve of the four fault blocks from which the gas storage is reconstructed is 478.0×10⁴ t. Data of the cumulative oil production and the cumulative water production in the oil reservoir development process is drawn in the semi-logarithmic coordinate system. The linear functional relation is determined to be within a data range of the cumulative oil production distributed in 1 million-1.15 million t through analysis, as shown in solid point data in FIG. 4. A formula lgWₚ=0.3448+0.0140×Nₚ is obtained through mathematical fitting of the segment of data. Wₚ denotes the cumulative water production, and Nₚ denotes the cumulative oil production. The formula is compared with a type-A water flooding curve formula lg(Wₚ+C)=A₁+B₁×Nₚ of oil reservoir water flooding development, such that A₁=0.3448 and B₁=0.0140 may be obviously obtained. The oil reservoir water drive recovery ratio is computed as 37.7% by substituting the numbers into a formula E_{w}=(1/B₁)×[lg(21.28/B₁−A₁)]/N.

S102, crude oil production extracted only due to a water flooding effect in the oil reservoir development process is determined according to the dynamic oil reservoir development data. Specifically,
computation is conducted on the basis of parameter data such as oil production, water production, water influx, and a crude oil property in the oil reservoir development process according to a formula.

$${N}_{pw} = {\sum }_{j = 1}^{n} {N}_{pj} \times [ ( {W}_{ej} + {W}_{ij} {B}_{wj} ) / ( {N}_{pj} {B}_{oj} + {W}_{pj} {B}_{wj} ) ] .$$

N_{pw} denotes crude oil production extracted only due to the water flooding effect in the oil reservoir development process. Further, j denotes different time points in monthly units in the oil reservoir development process. Further, n denotes a total development and extraction month number before the gas storage is reconstructed from the oil reservoir. Nₚⱼ, Wₑⱼ, Wᵢⱼ and Wₚⱼ denote oil production, natural water influx, an artificial water injection amount and water production in different months, respectively. B_{wj} and Bₒⱼ denote a formation water volume coefficient and a crude oil formation volume factor under average formation pressure in different months, respectively.

In a specific application process, the oil production Nₚⱼ, the artificial water injection amount Wᵢⱼ and the water production Wₚⱼ in different months may be accurately obtained according to actual production records. The natural water influx may be obtained through conventional oil reservoir engineering computation or numerical simulation according to the dynamic oil reservoir development data. The formation water volume coefficient B_{wj} and the crude oil formation volume factor Bₒⱼ may be obtained according to laboratory fluid tests.

FIG. 5 shows production data (such as oil production, water production, a water injection amount) recorded in a G oil reservoir development process in monthly units. Crude oil production extracted only due to the water flooding effect in a development process of the four fault blocks from which the gas storage is reconstructed is obtained as 37.4×10⁴ t through computation month by month and accumulation according to a formula

$${N}_{pw} = {\sum }_{j = 1}^{n} {N}_{pj} \times [ ( {W}_{ej} + {W}_{ij} {B}_{wj} ) / ( {N}_{pj} {B}_{oj} + {W}_{pj} {B}_{wj} ) ] .$$

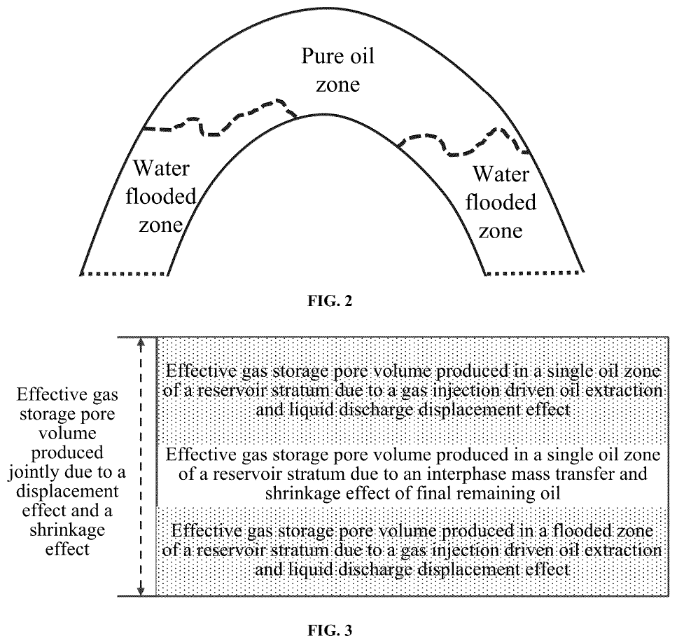

S103, an oil-bearing pore volume corresponding to a flooded zone of a reservoir influenced due to the water flooding effect in an original state before the oil reservoir is put into development when the gas storage is reconstructed from the flooded oil reservoir is determined according to the determined oil reservoir water drive recovery ratio and crude oil production extracted only due to the water flooding effect (as shown in FIG. 2). Specifically,
the oil-bearing pore volume V_{wz} corresponding to the flooded zone of the reservoir influenced due to the water flooding effect in the original state before the oil reservoir is put into development when the gas storage is reconstructed from the flooded oil reservoir is computed according to a formula V_{wz}=(N_{pw}×Bₒᵢ)/E_{w}. Bₒᵢ denotes a crude oil formation volume factor under original formation pressure of the oil reservoir.

In the embodiment of the present disclosure, the flooded zone of the reservoir refers to a zone influenced by water in the reservoir of the oil reservoir due to natural water invasion or artificial water injection when the gas storage is reconstructed from the flooded oil reservoir. Because oil reservoir development generally involves edge and bottom water injection and/or natural edge and bottom water invasion, the flooded zone of the reservoir is generally located in middle or bottom position of an oil reservoir structure. The lower a reservoir structure, the higher the flooding degree, and the greater the corresponding water saturation. Due to influence of reservoir heterogeneity and mobility contrast between oil and water, natural invasion and/or water injection may suddenly cause rush along a local high-permeability zone in the oil reservoir development process. That is, invaded water does not sweep all oil-bearing pore in the reservoir of the oil reservoir. In this case, a water invasion front edge is uneven and regular, a shape of the flooded zone of the oil reservoir in a three-dimensional space is irregular, and a shape of an interface between the flooded zone of the reservoir and the single oil zone not influenced by water invasion in middle and high parts of the reservoir of the oil reservoir is irregular, as shown in FIG. 2. Generally, through saturation logging and other technical means, according to indexes such as water saturation of the reservoir before the gas storage is reconstructed from the oil reservoir, the reservoir is divided into the flooded zone (having water saturation greater than irreducible water saturation) and the single oil zone (having water saturation equal to irreducible water saturation). Due to influence of various factors such as reservoir heterogeneity, complex oil-water distribution and well logging interpretation adaptability, logging interpretation accuracy of reservoir water saturation is quite uncertain. The denser and more heterogeneous the reservoir, the lower the interpretation accuracy of the reservoir water saturation after flooding of the oil reservoir, which makes it more difficult to identify the flooded zone and the single oil zone of the reservoir, and makes the error of the later-computed oil-bearing pore volume of each zone under the original formation pressure before oil reservoir development increasingly larger.

The oil-bearing pore volume V_{wz} corresponding to the flooded zone of the reservoir influenced due to the water flooding effect in the original state before the oil reservoir is put into development when the gas storage is reconstructed from the flooded oil reservoir is computed according to a formula V_{wz}=(N_{pw}×Bₒᵢ)/E_{w}. Water flooding efficiency E_{w} obtained through inverse computation on the basis of the dynamic oil reservoir development data is used and represents an overall sweep and displacement ratio of invaded water to the crude oil in the pore in the reservoir in the three-dimensional space in an oil reservoir water drive development process. A size of the three-dimensional space swept by natural gas water invasion and/or artificial water injection in the flooded oil reservoir is just reflected, and the flooded zone of the reservoir is accurately determined.

In Table 1, on the basis of the dynamic oil reservoir development data of the G water-flooded volatile oil reservoir and the computed parameters such as crude oil production extracted only due to the water flooding effect and the water flooding efficiency, according to a formula V_{wz}=(N_{pw}×Bₒᵢ)/E_{w}, the oil-bearing pore volume V_{wz} corresponding to the flooded zone of the reservoir in the original state before the oil reservoir is put into development is computed as 326.0×10⁴ m³.

Table 1 Computation results of an oil-bearing pore volume of a flooded zone of a reservoir in an original state before an oil reservoir is developed

| Crude oil | Density | Crude oil | Crude oil | | Oil-bearing pore |
|---|---|---|---|---|---|
| production | when | production | formation volume | Oil | volume of a flooded |
| extracted only | crude oil is | extracted only | factor under | reservoir | zone of a reservoir in |
| due to a water | on the | due to a water | original formation | water drive | an original state |
| flooding effect | ground | flooding effect | pressure of an oil | recovery | before an oil reservoir |
| (10⁴t) | (g/cm³) | (10⁴ m³) | reservoir (m³/m³) | ratio (%) | is developed (10⁴ m³) |
| 37.4 | 0.81 | 46.2 | 2.66 | 37.7 | 326.0 |

S104, an oil-bearing pore volume of a single oil zone of a reservoir not influenced due to the water flooding effect in an original state before the oil reservoir is put into development when the gas storage is reconstructed from the oil reservoir is determined according to a total original oil-bearing pore volume before the oil reservoir is put into development and the oil-bearing pore volume of the flooded zone of the reservoir in the original state before the oil reservoir is put into development (as shown in FIG. 2). Specifically,
the oil-bearing pore volume V_{oz} corresponding to the single oil zone of the reservoir not influenced due to the water flooding effect in the original state before the oil reservoir is put into development when the gas storage is reconstructed from the flooded oil reservoir is computed according to a formula V_{oz}=N×Bₒᵢ−V_{wz}.

In the embodiment of the present disclosure, the single oil zone of the reservoir refers to a zone not influenced by natural water invasion or artificial water injection when the gas storage is reconstructed from the flooded oil reservoir, and is generally located in middle and high parts of an oil reservoir structure. A shape of the single oil zone in a three-dimensional space is irregular, and a shape of an interface between the single oil zone of the reservoir and the flooded zone of the reservoir located in the middle and bottom positions of the reservoir is also irregular (as shown in FIG. 2). A distribution range and the oil-bearing pore volume of the single oil zone of the reservoir are expressed by the computed oil-bearing pore volume V_{oz} in the original state before the oil reservoir is put into development. N×Bₒᵢ in a formula V_{oz}=N×Bₒᵢ−V_{wz} computes a total original oil-bearing pore volume of the reservoir before the oil reservoir is put into development, and the oil-bearing pore volume of the flooded zone of the reservoir in the original state before the reservoir is put into development is subtracted from the total original oil-bearing pore volume, such that the oil-bearing pore volume of the single oil zone of the reservoir in the original state before the oil reservoir is put into development may be obtained. In this way, the total original oil-bearing pore volume of the reservoir remains unchanged before the oil reservoir is put into development.

Table 2 shows computation results of the oil-bearing pore volume of the single oil zone of the reservoir of the G water-flooded volatile oil reservoir in the original state before the oil reservoir is put into development. According to the dynamic reserves of the crude oil of four fault blocks of the gas storage planned, the original oil-bearing pore volume of the reservoir is computed as 1569.7×10⁴m³. Then, the oil-bearing pore volume 326.0×10⁴ m³ of the flooded zone of the reservoir in the original state before the oil reservoir is developed is subtracted from the original oil-bearing pore volume. In this way, the oil-bearing pore volume of the single oil zone of the reservoir in the original state before the oil reservoir is developed is obtained as 1243.7×10⁴ m³.

Table 2 Computation results of an oil-bearing pore volume of a single oil zone of a reservoir in an original state before an oil reservoir is developed

| | Density | | Crude oil formation | | Oil-bearing pore volume |
|---|---|---|---|---|---|
| Dynamic oil | when | Dynamic oil | volume factor | Original | of a single oil zone of a |
| reservoir | crude oil | reservoir | under original | oil-bearing pore | reservoir in an original |
| crude oil | is on the | crude oil | formation pressure | volume of a | state before an oil |
| reserve | ground | reserve | of an oil reservoir | reservoir of an oil | reservoir is developed |
| (10⁴t) | (g/cm³) | (10⁴ m³) | (m³/m³) | reservoir (10⁴ m³) | (10⁴ m³) |
| 478.0 | 0.81 | 590.1 | 2.66 | 1569.7 | 1243.7 |

S105, an effective pore volume for gas storage produced in the flooded zone and the single oil zone of the reservoir due to a gas injection driven oil recovery and liquid discharge displacement effect is determined according to macro sweep coefficients and micro displacement efficiency of injected gas in the flooded zone and the single oil zone of the reservoir separately when the gas storage is reconstructed from the oil reservoir (as shown in FIG. 3).

The effective pore volume V_{ged} for gas storage produced in the flooded zone and the single oil zone of the reservoir due to the gas injection driven oil recovery and liquid discharge displacement effect is computed according to a formula V_{ged}=V_{wz}×η_{wz}×E_{gwz}+V_{oz}×η_{oz}×E_{goz}. η_{wz} and η_{oz} denote the macro sweep coefficients of the injected gas in the flooded zone and the single oil zone of the reservoir when the gas storage is reconstructed from the flooded oil reservoir, respectively. E_{gwz} and E_{goz} denote the micro displacement efficiency of the injected gas in the flooded zone and the single oil zone when the gas storage is reconstructed from the flooded oil reservoir, respectively.

In the embodiment of the present disclosure, η_{wz} and η_{oz} may be obtained through the following method:
η_{wz} and η_{oz} are obtained by establishing a three-dimensional numerical simulation model representing features of the oil reservoir through software of Petrel RE or Eclipse according to geological features and dynamic development data of the flooded oil reservoir, and designing and conducting numerical simulation of the gas storage reconstructed from the flooded oil reservoir, or may be obtained according to an on-site pilot gas injection test of the gas storage.

Specifically,

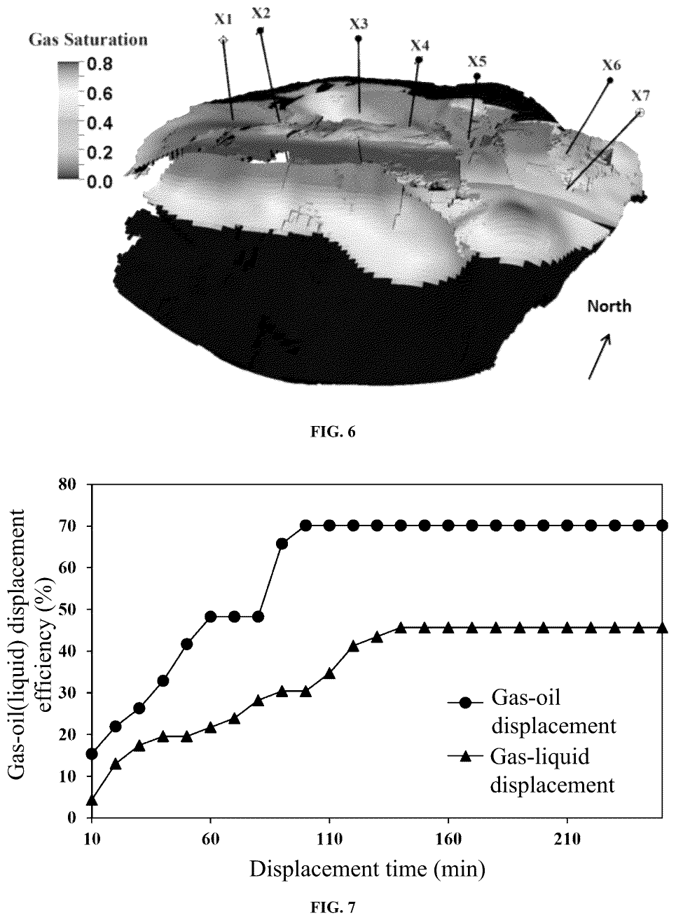

η_{wz} and η_{oz} are obtained by establishing the three-dimensional numerical simulation model representing the features of the oil reservoir through the software of Petrel RE or Eclipse and conducting numerical simulation as follows:
(1) According to data such as research results of oil reservoir geology, various dynamic development data and monitoring data, parameter adjustment and history matching are conducted on the numerical simulation model of the entire oil reservoir or a typical well group, and the three-dimensional numerical simulation model that may accurately reflect dynamic oil reservoir development is established. FIG. 6 shows a three-dimensional numerical simulation model of an entire G water-flooded volatile oil reservoir according to an embodiment of the present disclosure.
(2) Injection numerical simulation is conducted. According to tracer migration in injected gas or changes of reservoir gas saturation and formation pressure, underground oil-bearing pore volumes V₁ and V₂ of the reservoir within a range influenced by gas injected into the flooded zone and the single oil zone of the reservoir, and volumes Q₁ and Q₂ of gas separately injected into the flooded zone and the single oil zone of the reservoir may be directly obtained.
(3) Underground volumes V_{1g} and V_{2g} corresponding to the gas injected into the flooded zone and the single oil zone of the reservoir are computed according to formulas V_{1g}=Q₁×B_{1g} and V_{2g}=Q₂×B_{2g}, respectively. B_{1g} and B_{2g} denote gas volume coefficients under formation pressure in the flooded zone and the single oil zone of the reservoir after injection is completed, respectively.
(4) The macro sweep coefficients η_{wz} and η_{oz} of the injected gas in the flooded zone and the single oil zone of the reservoir are computed according to formulas η_{wz}=V_{1g}/V₁ and η_{oz}=V_{2g}/V₂, respectively.

A method for obtaining η_{wz} and η_{oz} according to the on-site pilot gas injection test of the gas storage is basically the same as the above numerical simulation, but is generally different in that suitable typical well groups are selected in the single oil zone of the reservoir and the flooded zone of the reservoir so as to conduct a storage reconstruction gas injection test.

In the embodiment of the present disclosure, numerical simulation is conducted on all the four fault blocks of the gas storage planned to be reconstructed from the G water-flooded volatile oil reservoir, and the macro sweep coefficients η_{wz} and η_{oz} of the injected gas in the flooded zone and the single oil zone of the reservoir are obtained as 64.8% and 69.1%.

In the embodiment of the present disclosure, E_{gwz} may be obtained through the following method:

According to geological features and dynamic development data of the flooded oil reservoir, a gas-liquid displacement experiment is designed, and gas-liquid displacement efficiency E_{gwz} of the flooded zone of the reservoir is tested through a gas displacement seepage experiment.
(1) A representative core in a flooded zone of a reservoir of a target oil reservoir is selected and made into standard plunger-shaped, a porosity and permeability of the core are measured after drying, and vacuum-pumping and pressurizing are sequentially conducted for full saturation, such that the target oil reservoir and formation water are simulated.
(2) Crude oil extracted from the target oil reservoir is regarded as a seepage medium for displacement, a constant-speed or constant-pressure oil-water displacement experiment is conducted, and displacement is conducted until no water is produced at an outlet end of the core. In this case, the core reaches an irreducible water (original saturated oil) state. The process reflects a function of forming an oil reservoir through early migration and accumulation of crude oil, and obtains a fluid occurrence state before the flooded oil reservoir is put into development. Through experimental measurement, the oil-bearing pore volume Vₒᵢ of the reservoir before the flooded oil reservoir is developed is obtained. It should be noted that irreducible water saturation reached by the core in the process has to be basically consistent with that of the reservoir of geological evaluation of the oil reservoir, such that representativeness of experimental results is ensured.
(3) A constant-speed or constant-pressure water flooding experiment is conducted, and displacement is conducted until no oil is produced at an outlet end of the core. In this case, the core reaches a water-residual oil displacement state. The process reflects a water flooding effect of the flooded zone of the reservoir in a flooded oil reservoir development process, and finally obtains a fluid occurrence state of the flooded zone of the reservoir before the gas storage is reconstructed from the flooded oil reservoir. It should be noted that irreducible water saturation reached by the core in the process has to be basically consistent with that of the reservoir of geological evaluation of the oil reservoir, such that representativeness of experimental results is ensured.
(4) Natural gas injected into the planned gas storage is regarded as a seepage medium for displacement, a constant-speed or constant-pressure gas-liquid displacement experiment is conducted, and displacement is conducted until no liquid (oil-water mixture) is produced at an outlet end of the core. In this case, the core reaches a gas-residual oil displacement state. The process reflects an oil recovery and liquid discharge displacement effect of gas injection of the gas storage reconstructed from the flooded oil reservoir on the flooded zone of the reservoir, and obtains a fluid occurrence state of the flooded zone of the reservoir after the gas storage is reconstructed from the flooded oil reservoir. A volume V_{Ld} of liquid (oil+water) displaced through gas injection driven oil recovery and liquid discharge may be obtained through experimental measurement.
(5) Gas-liquid displacement efficiency E_{gwz} of the flooded zone of the reservoir is computed according to a formula E_{gwz}=V_{Ld}/Vₒᵢ.

In the embodiment of the present disclosure, a core gas-liquid displacement experiment is conducted on the flooded zone of the reservoir of the G water-flooded volatile oil reservoir. According to experimental results, the gas-liquid displacement efficiency E_{gwz}of the flooded zone of the reservoir is obtained as 45.7%, as shown in FIG. 7.

In the embodiment of the present disclosure, E_{goz} may be obtained through the following method:

According to geological features and dynamic development data of the flooded oil reservoir, a gas-oil displacement experiment is designed, and gas-oil displacement efficiency E_{goz} of the single oil zone of the reservoir is tested through a gas displacement seepage experiment.

(1) A representative core in a single oil zone of a reservoir of a target oil reservoir is selected and made into standard plunger-shaped, a porosity and permeability of the core are measured after drying, and vacuum-pumping and pressurizing are sequentially conducted for full saturation, such that the target oil reservoir and formation water are simulated.

(2) Crude oil extracted from the target oil reservoir is regarded as a seepage medium for displacement, a constant-speed or constant-pressure oil-water displacement experiment is conducted, and displacement is conducted until no water is produced at an outlet end of the core. In this case, the core reaches an irreducible water (original saturated oil) state. The process reflects a function of forming an oil reservoir through early migration and accumulation of crude oil, and obtains a fluid occurrence state before the flooded oil reservoir is put into development. Through experimental measurement, the oil-bearing pore volume Vₒᵢ of the reservoir before the flooded oil reservoir is developed is obtained. It should be noted that irreducible water saturation reached by the core in the process has to be basically consistent with that of the reservoir of geological evaluation of the oil reservoir, such that representativeness of experimental results is ensured.

(3) Natural gas injected into the planned gas storage is regarded as a seepage medium for displacement, a constant-speed or constant-pressure gas-oil displacement experiment is conducted, and displacement is conducted until no oil is produced at an outlet end of the core. In this case, the core reaches a gas-residual oil displacement state. The process reflects a gas-oil displacement effect of storage reconstruction in the single oil zone of the reservoir in a flooded oil reservoir development process, and finally obtains a fluid occurrence state of the single oil zone of the reservoir before the gas storage is reconstructed from the flooded oil reservoir. A volume V_{od} of oil displaced through gas injection driven oil recovery may be obtained through experimental measurement.

(4) Gas-liquid displacement efficiency E_{goz} of the flooded zone of the reservoir is computed according to a formula E_{goz}=V_{od}/Vₒᵢ.

In the embodiment of the present disclosure, a core gas-oil displacement experiment is conducted on the single oil zone of the reservoir of the G water-flooded volatile oil reservoir. According to experimental results, the gas-oil displacement efficiency E_{goz} of the single oil zone of the reservoir is obtained as 70.2%, as shown in FIG. 7.

In the embodiment of the present disclosure, for the G water-flooded volatile oil reservoir, the oil-bearing pore volumes of the flooded zone and the single oil zone of the reservoir in the original state before the oil reservoir is put into development, and the macro sweep coefficients and the micro displacement efficiency of the injected gas of storage reconstruction in the above two zones are determined through the above steps respectively. In this way, the effective pore volume for gas storage produced in the flooded zone and the single oil zone of the reservoir due to the gas injection driven oil recovery and liquid discharge displacement effect may be determined as 699.6×10⁴ m³ according to a formula V_{ged}=V_{wz}×η_{wz}×E_{gwz}+V_{oz}×η_{oz}×E_{goz}, as shown in Table 3.

Table 3 Computation results of an effective pore volume for gas storage due to a displacement effect

| | Oil-bearing | Oil-bearing | | | | | Effective pore |
|---|---|---|---|---|---|---|---|
| | pore volume | pore volume | | | | | volume for |
| | of a flooded | of a single | Macro | Macro | | | gas storage |
| Original | zone of a | oil zone of a | sweep | sweep | Micro | Micro | due to a gas |
| oil-bearing | reservoir in | reservoir in | coefficient | coefficient | displacement | displacement | injection |
| pore | an original | an original | of injected | of injected | efficiency of | efficiency of | driven oil |
| volume of | state before | state before | gas in a | gas in a | injected gas | injected gas | recovery and |
| a reservoir | an oil | an oil | flooded | single oil | in a flooded | in a single oil | liquid discharge |
| of an oil | reservoir is | reservoir is | zone of a | zone of a | zone of a | zone of a | displacement |
| reservoir | developed | developed | reservoir | reservoir | reservoir | reservoir | effect |
| (10⁴ m³) | (10⁴ m³) | (10⁴ m³) | (%) | (%) | (%) | (%) | (10⁴ m³) |
| 1569.7 | 326.0 | 1243.7 | 64.8 | 69.1 | 45.7 | 70.2 | 699.6 |

S106, an effective pore volume for gas storage produced in the single oil zone of the reservoir of the gas storage reconstructed from the oil reservoir due to a volume shrinkage effect of final remaining oil is determined according to crude oil property change features caused by multi-cycle injection-production interphase mass transfer of the gas storage reconstructed from the oil reservoir.

The effective pore volume for gas storage produced in the single oil zone of the reservoir of the gas storage reconstructed from the oil reservoir due to a volume shrinkage effect of final remaining oil is computed according to a formula V_{ges}=V_{oz}×(1−η_{oz}×E_{oz})×(1−B_{os_max}/B_{oi_max}). V_{ges} denotes the effective pore volume for gas storage added in the single oil zone of the reservoir of the gas storage reconstructed from the oil reservoir due to the volume shrinkage effect of the final remaining oil. V_{oz} denotes the oil-bearing pore volume of the single oil zone of the reservoir not influenced due to the water flooding effect in the original state before the oil reservoir is put into development when the gas storage is reconstructed from the oil reservoir. η_{oz} denotes the macro sweep coefficient of the injected gas in the single oil zone of the reservoir when the gas storage is reconstructed from the oil reservoir. E_{oz} denotes the micro displacement efficiency of the injected gas in the single oil zone of the reservoir when the gas storage is reconstructed from the oil reservoir. B_{os_max} denotes a volume coefficient of the final remaining oil in the single oil zone of the reservoir under designed upper limit pressure of the gas storage after circulating injection-production of the gas storage reconstructed from the oil reservoir is stabilized. B_{oi_max} denotes a volume coefficient of original crude oil of the reservoir of the oil reservoir under the designed upper limit pressure of the gas storage.

Interphase mass transfer refers to a complex component exchange behavior between the injected gas (generally dry gas greater in methane content) and crude oil of the reservoir during multi-cycle injection-production of the gas storage reconstructed from the oil reservoir, which is mainly manifested in continuous extraction of intermediate hydrocarbons greater in content in the crude oil by the injected gas. In this way, contents of heavy components such as C7+ in the crude oil of the reservoir increase constantly, leading to increase in crude oil density and volume shrinkage. Crude oil distribution features and the effective gas storage pore in the reservoir are changed.

When the gas storage is reconstructed from the water-flooded volatile oil reservoir, the injected gas is generally concentrated in the middle and high parts of the structure, and has a large contact area with the crude oil in the single oil zone of the reservoir. In addition, only two phase fluids of gas and oil exist in pore of rocks of the reservoir in the zone. Therefore, an interphase mass transfer effect of the single oil zone of the reservoir is intense, properties of the crude oil change significantly, and a volume of the crude oil shrinks greatly. However, a contact area between the injected gas and the crude oil in the flooded zone of the reservoir at the middle and bottom positions of the structure is small, and three phase fluids of gas, oil and water exist in the pore of the rocks of the reservoir in the zone. Because the flooded zone of the reservoir is in a residual oil saturation state after water displacement is conducted on the oil reservoir, the interphase mass transfer effect of the flooded zone of the reservoir is poor. In specific application, the effective pore volume for gas storage added in the flooded zone of the reservoir of the gas storage reconstructed from the oil reservoir due to the volume shrinkage effect of the final remaining oil may be ignored.

In the embodiment of the present disclosure, B_{os_max} may be obtained through the following method:

A numerical simulation or physical simulation experiment of circulating injection-production phase balance of crude oil and injected gas is designed according to properties of the crude oil and the injected gas of the reservoir of the gas storage reconstructed from the oil reservoir and a circulating injection-production operation condition of the gas storage, and the volume coefficient B_{os_max} of the final remaining oil in the single oil zone of the reservoir under the designed upper limit pressure of the gas storage after circulating injection-production of the gas storage reconstructed from the oil reservoir is stabilized is obtained through simulation or testing.

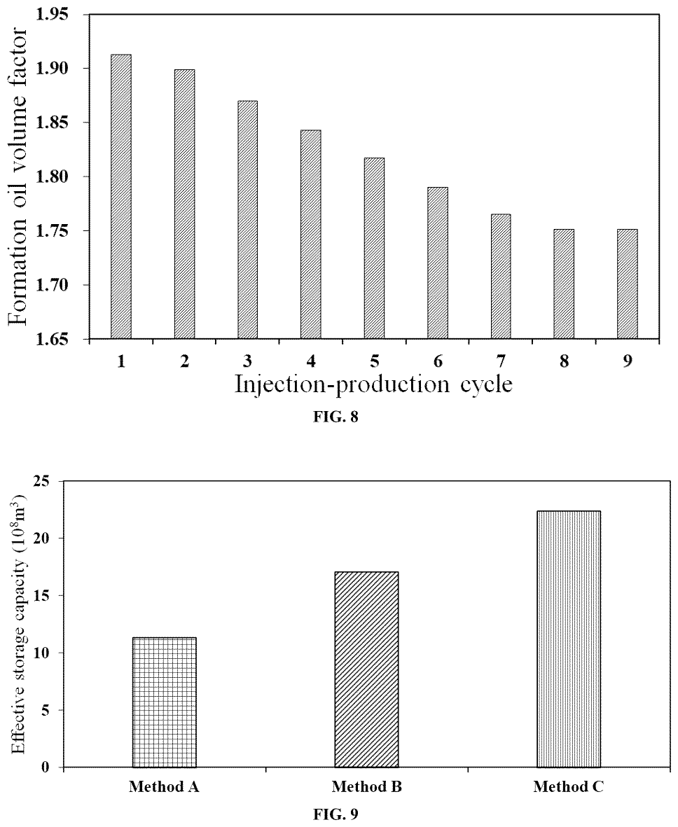

In a process of a numerical simulation or physical simulation experiment, a change law of the volume coefficient B_{os_max} of remaining oil in the single oil zone of the reservoir in different injection-production cycles under the designed upper limit pressure of the gas storage is analyzed. When B_{os_max} no longer changes with increase of the injection-production cycle of the gas storage, or a change degree may be ignored (for example, a change degree between crude oil formation volume factors in first and second injection-production cycles is smaller than 10%), the coefficient is considered as the volume coefficient after circulating injection-production of the gas storage reconstructed from the oil reservoir is stabilized, as shown in FIG. 8.

In the embodiment of the present disclosure, for the G water-flooded volatile oil reservoir, samples of the crude oil are extracted from the oil reservoir, a physical simulation experiment of phase balance of injected dry gas and crude oil is conducted, and the volume coefficient B_{os_max} of the crude oil in different injection-production cycles under the designed upper limit pressure of the gas storage is obtained through testing, as shown in Table 4. As may be seen from Table 4, due to an interphase mass transfer effect between the injected dry gas and the crude oil when the gas storage is reconstructed from the water-flooded volatile oil reservoir, the injected dry gas continuously extracts the intermediate hydrocarbons in the crude oil, which makes the crude oil gradually approach conventional black oil in property, a density of the crude oil increases, and accordingly, the volume coefficient continuously decreases. After 8 injection-production cycles in Table 4, the volume coefficient of the final remaining oil in the single oil zone of the reservoir is 1.751 under the designed upper limit pressure of the gas storage.

Table 4 Experimental results of a volume coefficient of final remaining oil in a single oil zone of a reservoir under designed upper limit pressure of a gas storage

| | Injection-production cycle | | | | | | | | |
|---|---|---|---|---|---|---|---|---|---|
| | 1 | 2 | 3 | 4 | 5 | 6 | 7 | 8 | 9 |
| Volume coefficient | 1.913 | 1.899 | 1.870 | 1.843 | 1.817 | 1.790 | 1.765 | 1.751 | 1.751 |
| of remaining oil | | | | | | | | | |

In the embodiment of the present disclosure, B_{oi_max} may be obtained through the following method:

The volume coefficient B_{oi_max} of the original crude oil of the reservoir of the oil reservoir under the designed upper limit pressure of the gas storage is obtained through a laboratory high-pressure physical property determination experiment or theoretical computation according to samples of crude oil fluid of a target oil reservoir.

In the embodiment of the present disclosure, for the G water-flooded volatile oil reservoir, a high-pressure physical parameter test is conducted on original crude oil of the reservoir of the oil reservoir in a laboratory, and the volume coefficient of the crude oil under the designed upper limit pressure of the gas storage is obtained as 2.66.

After the above parameters are determined, the effective pore volume for gas storage added in the single oil zone of the reservoir of the gas storage reconstructed from the oil reservoir due to the volume shrinkage effect of the final remaining oil may be computed as 218.9×10⁴ m³ according to a formula V_{ges}=V_{oz}×(1−η_{oz}×E_{oz})×(1−B_{os_max}/B_{oi_max}) as shown in Table 5.

Table 5 Computation results of an effective pore volume for gas storage added in a single oil zone of a reservoir due to a volume shrinkage effect of final remaining oil

| | | | Volume | | |
|---|---|---|---|---|---|
| Oil-bearing | | | coefficient | | Effective pore |
| pore volume of | | | of final | Volume | volume for gas |
| a single oil | | Micro | remaining oil in | coefficient of | storage added in |
| zone of a | | displacement | a single oil zone | original crude | a single oil zone |
| reservoir in an | Macro sweep | efficiency of | of a reservoir | oil of a | of a reservoir due |
| original state | coefficient of | injected gas | under designed | reservoir under | to a volume |
| before an oil | injected gas in a | in a single oil | upper limit | designed upper | shrinkage effect |
| reservoir is | single oil zone | zone of a | pressure of a | limit pressure of | of final |
| developed | of a reservoir | reservoir | gas storage | a gas storage | remaining oil |
| (10⁴ m³) | (%) | (%) | (Dimensionless) | (Dimensionless) | (10⁴ m³) |
| 1243.7 | 69.1 | 70.2 | 1.751 | 2.66 | 218.9 |

S107, the effective storage capacity of the gas storage reconstructed from the water-flooded volatile oil reservoir is computed according to a total effective pore volume for gas storage produced due to the gas injection driven oil recovery and liquid discharge displacement effect and the volume shrinkage effect of the final remaining oil in the single oil zone of the reservoir. Specifically,

the total effective pore volume V_{get} for gas storage added due to the gas injection driven oil recovery and liquid discharge displacement effect and the volume shrinkage effect of the final remaining oil in the single oil zone is computed according to a formula V_{get}=V_{ged}+V_{ges}; and

the effective storage capacity of the gas storage reconstructed from the flooded oil reservoir is computed according to a formula Iₑₘₐₓ=V_{get}/B_{dgmax}. B_{dgmax} denotes a volume coefficient of the injected gas under the designed upper limit pressure of the gas storage.

V_{get} denotes the total effective pore volume for gas storage produced due to the gas injection driven oil recovery and liquid discharge displacement effect and the volume shrinkage effect of the final remaining oil in the single oil zone of the reservoir. V_{ged} denotes the effective pore volume for gas storage produced in the flooded zone and the single oil zone of the reservoir due to the gas injection driven oil recovery and liquid discharge displacement effect. V_{ges} denotes the effective pore volume for gas storage produced in the single oil zone of the reservoir due to the volume shrinkage effect of the final remaining oil caused by crude oil property changes. B_{dgmax} denotes a volume coefficient of the injected gas under the designed upper limit pressure of the gas storage.

In the embodiment of the present disclosure, when the gas storage is reconstructed from the G water-flooded volatile oil reservoir, the total effective pore volume and the effective storage capacity are computed as 918.5×10⁴ m³ and 22.40×10⁸ m³, respectively, as shown in Table 6.

Table 6 Computation results of a total effective pore volume and effective storage capacity of a gas storage reconstructed from a G water-flooded volatile oil reservoir

| Effective pore volume | Effective pore | | | |
|---|---|---|---|---|
| for gas storage | volume for gas | Effective pore | | |
| produced due to a gas | storage | volume for gas | | Effective storage |
| injection driven oil | produced due to | storage produced | Volume coefficient of | capacity of a gas |
| recovery and liquid | a shrinkage | due to a | injected gas under | storage |
| discharge | effect of | displacement effect | designed upper limit | reconstructed from |
| displacement effect | remaining oil | and a shrinkage | pressure of a gas | a flooded oil |
| (10⁴ m³) | (10⁴ m³) | effect (10⁴ m³) | storage (Dimensionless) | reservoir (10⁸ m³) |
| 699.6 | 218.9 | 918.5 | 0.0041 | 22.40 |

Effects and advantages of the new method of the present disclosure are compared with those of the existing method as follows:

FIGS. 7 and 9 show comparison results of effective storage capacity of a gas storage reconstructed from a G water-flooded volatile oil reservoir computed through the existing method and the new method of the present disclosure.

Table 7 Results of effective storage capacity of a gas storage reconstructed from a G water-flooded volatile oil reservoir computed through different methods

| | | Computation | |
|---|---|---|---|
| | | results of | |
| | | effective | |
| | | storage | |
| Method | | capacity | Error |
| Name | Advantages and disadvantages of methods | (10⁸ m³) | (%) |
| Method | Not consider different fluid zones of a | 11.33 | 49.4 |
| A | reservoir and interphase mass transfer | | |
| Method | Consider different fluid distributions of a | 17.06 | 23.8 |
| B | reservoir and not consider interphase mass | | |
| | transfer | | |
| Method | Consider both different fluid zones of a | 22.40 | — |
| C | reservoir and interphase mass transfer | | |

An existing method only for designing effective storage capacity of a gas storage reconstructed from a conventional completely-flooded black oil reservoir (referred to as method A) is used, and the effective storage capacity is computed as 11.33×10⁸ m³. Because the method considers neither a difference of utilization efficiency of gas injection driven oil displacement and liquid discharge of different fluid zones of the reservoir when the gas storage is reconstructed from the water-flooded volatile oil reservoir, nor the effective gas storage space added by the volume shrinkage effect of the remaining oil in the reservoir caused by interphase mass transfer, the evaluated effective storage capacity is the smallest, which seriously underestimates abilities of gas storage and peak regulation of a gas storage reconstructed from a volatile oil reservoir that is not completely flooded, and brings severe challenges to site selection and evaluation, engineering design and economic benefit evaluation of storage reconstruction.

When method considers the difference of utilization efficiency of gas injection driven oil displacement and liquid discharge of different fluid zones of the reservoir when the gas storage is reconstructed from the water-flooded volatile oil reservoir, that is, when the method of the present disclosure is used to accurately distinguish the flooded zone and the single oil zone of the reservoir and determine the difference of gas injection driven utilization efficiency between the flooded zone and the single oil zone, regardless of the effective gas storage space (referred to as method B) added by the volume shrinkage effect of the remaining oil of the reservoir caused by interphase mass transfer, the effective storage capacity is computed as 17.06×10⁸ m³. The effective storage capacity of the gas storage evaluated through the method is intermediate, but still underestimates the abilities of gas storage and peak regulation of the gas storage reconstructed from the volatile oil reservoir that is not completely flooded. The more volatile the oil reservoir, the lower the degree of flooding, and the larger the error of the effective storage capacity of the gas storage computed through the method.

The new method (referred to as method C) provided by the present disclosure is used. The method considers not only different fluid zones of the reservoir when the gas storage is reconstructed from the water-flooded volatile oil reservoir, but also the difference of utilization efficiency of gas injection driven oil displacement and liquid discharge and the effective gas storage space added by the volume shrinkage effect of the remaining oil of the reservoir caused by interphase mass transfer, the effective storage capacity is computed as 22.40×10⁸ m³. The new method starts from an internal mechanism of multiphase fluid seepage and phase behaviors caused by gas injection driven oil recovery and liquid discharge gas storage space utilization and storage capacity formation in the gas storage reconstructed from the water-flooded volatile oil reservoir, and meanwhile considers influence of different fluid zones of the reservoir during gas injection of reconstruction of the gas storage and their difference of gas injection driven utilization efficiency (macro sweep coefficients and micro displacement efficiency of the injected gas) and changes of properties of the final remaining oil of the reservoir caused by multi-cycle injection-production injected gas-crude oil interphase mass transfer on the effective gas storage space. The factors considered are more comprehensive, and are all suitable for predicting the effective storage capacity of the gas storage reconstructed from conventional black oil and volatile oil reservoirs, such that computation errors of the effective storage capacity are greatly reduced. Moreover, the more volatile the oil reservoir, the lower the degree of flooding, and the higher computation accuracy of the new method for the effective storage capacity of the gas storage.

What is claimed is:

1. A method for determining effective storage capacity of a gas storage reconstructed from a water-flooded volatile oil reservoir, comprising the following steps:
step 1: determining an oil reservoir water drive recovery ratio according to dynamic oil reservoir development data;
step 2: determining, according to the dynamic oil reservoir development data, crude oil production extracted only due to a water flooding effect in an oil reservoir development process;
step 3: determining an oil-bearing pore volume of a flooded zone of a reservoir influenced due to the water flooding effect in an original state before an oil reservoir is put into development when the gas storage is reconstructed from the oil reservoir according to the oil reservoir water drive recovery ratio and crude oil production extracted only due to the water flooding effect;
step 4: determining an oil-bearing pore volume of a single oil zone of a reservoir not influenced due to the water flooding effect in an original state before the oil reservoir is put into development when the gas storage is reconstructed from the oil reservoir according to a total original oil-bearing pore volume before the oil reservoir is put into development and the oil-bearing pore volume of the flooded zone of the reservoir in the original state before the oil reservoir is put into development;
step 5: determining an effective pore volume for gas storage produced in the flooded zone and the single oil zone of the reservoir due to a gas injection driven oil recovery and liquid discharge displacement effect according to macro sweep coefficients and micro displacement efficiency of injected gas in the flooded zone and the single oil zone of the reservoir separately when the gas storage is reconstructed from the oil reservoir;
step 6: determining an effective pore volume for gas storage produced in the single oil zone of the reservoir of the gas storage reconstructed from the oil reservoir due to a volume shrinkage effect of final remaining oil according to crude oil property change features caused by multi-cycle injection-production interphase mass transfer of the gas storage reconstructed from the oil reservoir; and
step 7: computing the effective storage capacity of the gas storage reconstructed from the water-flooded volatile oil reservoir according to a total effective pore volume for gas storage produced due to the gas injection driven oil recovery and liquid discharge displacement effect and the volume shrinkage effect of the final remaining oil in the single oil zone of the reservoir.

2. The method for determining effective storage capacity of a gas storage reconstructed from a water-flooded volatile oil reservoir according to claim 1, wherein the determining an oil reservoir water drive recovery ratio in step 1 comprises:
conducting computation on the basis of data of oil production and water production in the oil reservoir development process according to a formula E_{w}=(1/B₁)×[lg(21.28/B₁−A₁)]/N, wherein
E_{w} denotes the oil reservoir water drive recovery ratio, A₁ and B₁ denote an intercept and a slope obtained through mathematical fitting of a linear functional relation between cumulative oil production and cumulative water production in an oil reservoir water drive development process in a semi-logarithmic coordinate system, respectively, and N denotes a dynamic reserve of the oil reservoir.

3. The method for determining effective storage capacity of a gas storage reconstructed from a water-flooded volatile oil reservoir according to claim 1, wherein the determining crude oil production extracted only due to a water flooding effect in an oil reservoir development process in step 2 comprises:
conducting computation on the basis of parameter data of oil production, water production, water influx, a water injection amount and a crude oil property in the oil reservoir development process according to a formula
$${N}_{pw} = {\sum }_{j = 1}^{n} {N}_{pj} \times [ ( {W}_{ej} + {W}_{ij} {B}_{wj} ) / ( {N}_{pj} {B}_{oj} + {W}_{pj} {B}_{wj} ) ] ,$$
N_{pw} denotes crude oil production extracted only due to the water flooding effect in the oil reservoir development process, j denotes different time points in monthly units in the oil reservoir development process, n denotes a total development and extraction month number before the gas storage is reconstructed from the oil reservoir, Nₚⱼ, Wₑⱼ, Wᵢⱼ and Wₚⱼ denote oil production, natural water influx, an artificial water injection amount and water production in different months, respectively, and B_{wj} and Bₒⱼ denote a formation water volume coefficient and a crude oil formation volume factor under average formation pressure in different months, respectively.

4. The method for determining effective storage capacity of a gas storage reconstructed from a water-flooded volatile oil reservoir according to claim 1, wherein the determining an oil-bearing pore volume of a flooded zone of a reservoir influenced due to the water flooding effect in an original state before an oil reservoir is put into development when the gas storage is reconstructed from the oil reservoir in step 3 comprises: conducting computation according to a formula V_{wz}=(N_{pw}×Bₒᵢ)/E wherein
V_{wz} denotes the oil-bearing pore volume of the flooded zone of the reservoir influenced due to the water flooding effect in the original state before the oil reservoir is put into development when the gas storage is reconstructed from the oil reservoir, N_{pw} denotes crude oil production extracted only due to the water flooding effect in an oil reservoir water drive development process, and Bₒᵢ denotes a crude oil formation volume factor under original formation pressure of the oil reservoir.

5. The method for determining effective storage capacity of a gas storage reconstructed from a water-flooded volatile oil reservoir according to claim 1, wherein the determining an oil-bearing pore volume of a single oil zone of a reservoir not influenced due to the water flooding effect in an original state before the oil reservoir is put into development when the gas storage is reconstructed in step 4 comprises: conducting computation according to a formula V_{oz}=N×Bₒᵢ−V_{wz}, wherein
V_{oz} denotes the oil-bearing pore volume of the single oil zone of the reservoir not influenced due to the water flooding effect in the original state before the oil reservoir is put into development when the gas storage is reconstructed from the oil reservoir, N denotes a dynamic reserve of the oil reservoir, Bₒᵢ denotes a crude oil formation volume factor under original formation pressure of the oil reservoir, and V_{wz} denotes the oil-bearing pore volume of the flooded zone of the reservoir influenced due to the water flooding effect in the original state before the oil reservoir is put into development when the gas storage is reconstructed from the oil reservoir.

6. The method for determining effective storage capacity of a gas storage reconstructed from a water-flooded volatile oil reservoir according to claim 1, wherein the determining an effective pore volume for gas storage produced in the flooded zone and the single oil zone of the reservoir due to a gas injection driven oil recovery and liquid discharge displacement effect in step 5 comprises: conducting computation according to a formula V_{ged}=V_{wz}×η_{wz}×E_{wz}+V_{oz}×η_{oz}×E_{oz} wherein
V_{ged} denotes the effective pore volume for gas storage produced in the flooded zone and the single oil zone of the reservoir due to the gas injection driven oil recovery and liquid discharge displacement effect, η_{wz} and η_{oz} denote the macro sweep coefficients of the injected gas in the flooded zone and the single oil zone of the reservoir when the gas storage is reconstructed from the oil reservoir, respectively, and E_{wz} and E_{oz} denote the micro displacement efficiency of the injected gas in the flooded zone and the single oil zone of the reservoir when the gas storage is reconstructed from the oil reservoir, respectively.

7. The method for determining effective storage capacity of a gas storage reconstructed from a water-flooded volatile oil reservoir according to claim 6, wherein the macro sweep coefficient η_{wz} of the flooded zone of the reservoir and the macro sweep coefficient η_{oz} of the single oil zone of the reservoir are computed by establishing a three-dimensional numerical simulation model of a target oil reservoir through software of Petrel RE or Eclipse and simulating a natural gas injection process of the gas storage reconstructed from the oil reservoir, or are obtained according to an on-site pilot gas injection test of the gas storage.

8. The method for determining effective storage capacity of a gas storage reconstructed from a water-flooded volatile oil reservoir according to claim 6, wherein the micro displacement efficiency of the flooded zone of the reservoir and the micro displacement efficiency of the single oil zone of the reservoir are obtained through the following steps:
designing and developing two displacement simulation experiments of gas injection driven oil displacement and liquid discharge and gas injection driven oil displacement of a core according to fluid distribution features of the flooded zone and the single oil zone of the reservoir when the gas storage is reconstructed from the oil reservoir, respectively, and separately obtaining the micro displacement efficiency E_{wz} of the flooded zone of the reservoir and the micro displacement efficiency E_{oz} of the single oil zone of the reservoir through analysis.

9. The method for determining effective storage capacity of a gas storage reconstructed from a water-flooded volatile oil reservoir according to claim 1, wherein the determining an effective pore volume for gas storage produced in the single oil zone of the reservoir of the gas storage reconstructed from the oil reservoir due to a volume shrinkage effect of final remaining oil in step 6 comprises: conducting computation according to a formula V_{ges}=V_{oz}×(1−η_{oz}×E_{oz})×(1−B_{os_max}/B_{oi_max}), wherein
V_{ges} denotes the effective pore volume for gas storage produced in the single oil zone of the reservoir of the gas storage reconstructed from the oil reservoir due to the volume shrinkage effect of the final remaining oil, V_{oz} denotes the oil-bearing pore volume of the single oil zone of the reservoir not influenced due to the water flooding effect in the original state before the oil reservoir is put into development when the gas storage is reconstructed from the oil reservoir, η_{oz} denotes the macro sweep coefficient of the injected gas in the single oil zone of the reservoir when the gas storage is reconstructed from the oil reservoir, E_{oz} denotes the micro displacement efficiency of the injected gas in the single oil zone of the reservoir when the gas storage is reconstructed from the oil reservoir, B_{os_max} denotes a volume coefficient of the final remaining oil in the single oil zone of the reservoir under designed upper limit pressure of the gas storage after circulating injection-production of the gas storage reconstructed from the oil reservoir is stabilized, and B_{oi_max} denotes a volume coefficient of original crude oil of the reservoir of the oil reservoir under the designed upper limit pressure of the gas storage;
the volume coefficient of the final remaining oil in the single oil zone of the reservoir under designed upper limit pressure of the gas storage after circulating injection-production of the gas storage reconstructed from the oil reservoir is stabilized is obtained through the following steps: designing a numerical simulation or physical simulation experiment of circulating injection-production phase balance of crude oil and injected gas according to properties of the crude oil and the injected gas of the reservoir of the gas storage reconstructed from the oil reservoir and a circulating injection-production operation condition of the gas storage, and obtaining the volume coefficient B_{os_max} of the final remaining oil in the single oil zone of the reservoir under the designed upper limit pressure of the gas storage after circulating injection-production of the gas storage reconstructed from the oil reservoir is stabilized through simulation or testing; and
the volume coefficient of the original crude oil of the reservoir of the oil reservoir under the designed upper limit pressure of the gas storage is obtained through the following step: obtaining the volume coefficient B_{oi_max} of the original crude oil of the reservoir of the oil reservoir under the designed upper limit pressure of the gas storage through a laboratory high-pressure physical property determination experiment or theoretical computation according to samples of crude oil fluid of a target oil reservoir.

10. The method for determining effective storage capacity of a gas storage reconstructed from a water-flooded volatile oil reservoir according to claim 1, wherein the computing the effective storage capacity of the gas storage reconstructed from the water-flooded volatile oil reservoir according to a determined total effective pore volume for gas storage produced due to the gas injection driven oil recovery and liquid discharge displacement effect and the volume shrinkage effect of the final remaining oil in the single oil zone of the reservoir in step 7 comprises:
computing the total effective pore volume V_{get} for gas storage produced due to the gas injection driven oil recovery and liquid discharge displacement effect and the volume shrinkage effect of the final remaining oil in the single oil zone of the reservoir according to a formula get ged ges; and
computing the effective storage capacity of the gas storage reconstructed from the oil reservoir according to a formula Iₑₘₐₓ=V_{get}/B_{dgmax}, wherein
V_{get} denotes the total effective pore volume for gas storage produced due to the gas injection driven oil recovery and liquid discharge displacement effect and the volume shrinkage effect of the final remaining oil in the single oil zone of the reservoir, V_{ged} denotes the effective pore volume for gas storage produced in the flooded zone and the single oil zone of the reservoir due to the gas injection driven oil recovery and liquid discharge displacement effect, V_{ges} denotes the effective pore volume for gas storage produced in the single oil zone of the reservoir due to the volume shrinkage effect of the final remaining oil caused by crude oil property changes, and B_{dgmax} denotes a volume coefficient of the injected gas under the designed upper limit pressure of the gas storage.
